(19) United States
(12) Patent Application Publication
(10) Pub. No.: US 2025/0302558 A1
(43) Pub. Date: Oct. 2, 2025

(54) SYSTEM AND METHODS FOR IMPROVING TELEOPERATIONAL CONTROL

(71) Applicant: Intuitive Surgical Operations, Inc., Sunnyvale, CA (US)

(72) Inventors: Kevin Supakkul, Mountain View, CA (US); Joseph Giallo, Cary, NC (US); Junjie Zhang, San Jose, CA (US); Kyle Petrich, Los Gatos, CA (US)

(73) Assignee: Intuitive Surgical Operations, Inc., Sunnyvale, CA (US)

(21) Appl. No.: 19/093,820

(22) Filed: Mar. 28, 2025

Related U.S. Application Data
(60) Provisional application No. 63/571,735, filed on Mar. 29, 2024.
(60) Provisional application No. 63/571,756, filed on Mar. 29, 2024.

(51) Int. Cl. A61B 34/35; A61B 34/00; A61B 90/00

(52) U.S. Cl. CPC A61B 34/35; A61B 34/25; A61B 34/70; A61B 90/37

(57) ABSTRACT

A computer-assisted system includes: a manipulator assembly configured to support a first instrument and an imaging device; a first input device configured to be manipulated by an operator relative to an input device coordinate frame; and a control system communicatively coupled to the first input device and the manipulator assembly. The control system is configured to: determine a first roll orientation of the imaging device; record the first roll orientation of the imaging device in response to enablement of a first teleoperational control session of the first instrument by the first input device. During the first teleoperational control session, the control system is configured to: determine a current roll orientation of the imaging device; determine a difference between the current roll orientation and the first roll orientation; determine whether or not the difference between the current roll orientation and the first roll orientation exceeds a first threshold.

### CROSS-REFERENCE TO RELATED APPLICATIONS

This Patent Application claims the benefit of priority under 35 U S.C. § 119(e) to U.S. Provisional Patent Application Ser. No. 63/571,756, filed on Mar. 29, 2024, which is hereby incorporated by reference herein in its entirety. This Patent Application claims the benefit of priority under 35 U S.C. § 119(e) to U.S. Provisional Patent Application Ser. No. 63/571,735, filed on Mar. 29, 2024, which is hereby incorporated by reference herein in its entirety.

### BACKGROUND

### Field of Invention

The present invention generally provides improved computer-assisted devices, systems, and methods.

### Overview

Computer-assisted systems can be used to perform a task at a workspace. For example, a computer-assisted system may comprise an input system (e.g., a console, a surgeon's console), a manipulator assembly, and a control system. For example, in a medical context, the manipulator assembly may be used to perform diagnosis, non-surgical treatment, surgical treatment (e.g., through minimally invasive apertures or natural orifices) and is guided by the user based on images or video from the imaging device.

As another example, a computer-assisted system may comprise a robotic system (e.g., industrial and recreational systems), and may include one or more robotic manipulators to manipulate instruments for performing the task.

The computer-assisted system can be automated, semi-automated, teleoperated, or any combination thereof. In any mode of operation, a high degree of coordination of the manipulator assembly is required. If coordination of the manipulator assembly is lost or degraded, operation of the computer-assisted system may result in unintended movements.

Therefore, an efficient, reliable, and/or easier-to-perform method of maintaining/evaluating coordination of the computer-assisted systems is, therefore, highly desirable.

### SUMMARY

In general, in one aspect, one or more embodiments relate to a computer-assisted system including: a manipulator assembly configured to support a first instrument and an imaging device; a first input device configured to be manipulated by an operator relative to an input device coordinate frame; and a control system communicatively coupled to the first input device and the manipulator assembly. The control system is configured to: determine a first roll orientation of the imaging device; record the first roll orientation of the imaging device in response to enablement of a first teleoperational control session of the first instrument by the first input device. During the first teleoperational control session, the control system is configured to: determine a first transformation between the input device coordinate frame and a workspace coordinate frame based on the first roll orientation of the imaging device; receive inputs from the first input device to cause the manipulator assembly to manipulate the first instrument relative to the workspace coordinate frame based on the first transformation; determine a current roll orientation of the imaging device; determine a difference between the current roll orientation and the first roll orientation; determine whether or not the difference between the current roll orientation and the first roll orientation exceeds a first threshold; and in response to the difference between the current roll orientation and the first roll orientation exceeding the first threshold, perform a mitigating action.

In general, in one aspect, one or more embodiments relate to method of operating a computer-assisted system including a first input device configured to be manipulated by an operator, a manipulator assembly configured to support a first instrument and an imaging device, and a control system communicatively coupled to the first input device and the manipulator assembly. The method comprises: determining a first roll orientation of the imaging device; recording the first roll orientation of the imaging device in response to enablement of a first teleoperational control session of the first instrument by the first input device. During the first teleoperational control session, the method includes: determining a first transformation between an input device coordinate frame and a workspace coordinate frame based on the first roll orientation of the imaging device; receiving inputs from the first input device to cause the manipulator assembly to manipulate the first instrument relative to the workspace coordinate frame based on the first transformation; determining a current roll orientation of the imaging device; determining a difference between the current roll orientation and the first roll orientation; determining whether or not the difference between the current roll orientation and the first roll orientation exceeds a first threshold; and in response to the difference between the current roll orientation and the first roll orientation exceeding the first threshold, performing a mitigating action.

Other aspects of the invention will be apparent from the following description and the appended claims.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a simplified diagram and schematic of an example computer-assisted system, in accordance with one or more embodiments.

FIG. 2A shows a user interface system, in accordance with one or more embodiments.

FIG. 2B is a perspective view of a controller portion 200 of an example input device 152 of the user interface system 120 shown in FIG. 2A.

FIGS. 2C-2D show examples of a manipulator assembly, in accordance with one or more embodiments.

FIG. 3 is a simplified diagram showing example coordinate frames relevant to a computer-assisted system, in accordance with one or more embodiments.

FIGS. 4A-4B show example fields of view of an imaging device, in accordance with one or more embodiments.

FIGS. 5A-5C show example fields of view of an imaging device, in accordance with one or more embodiments.

FIG. 6 shows a method according to one or more embodiments.

FIGS. 7A-7C show methods according to one or more embodiments.

FIG. 8 shows a method according to one or more embodiments.

### DETAILED DESCRIPTION

Specific embodiments of the disclosure will now be described in detail with reference to the accompanying figures. Like elements in the various figures are denoted by like reference numerals for consistency.

In the following detailed description of embodiments of the disclosure, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements, and is not to limit any clement to being only a single clement unless expressly disclosed, such as by the use of the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

This disclosure describes various devices, elements, and portions of computer-assisted systems and elements in terms of their state in three-dimensional space. As used herein, the term “position” refers to the location of an element or a portion of an element (e.g., three degrees of translational freedom in a three-dimensional space, such as along Cartesian x-, y-, and z-coordinates). As used herein, the term “orientation” refers to the rotational placement of an element or a portion of an element (e.g., three degrees of rotational freedom in three-dimensional space, such as about roll, pitch, and yaw axes, represented in angle-axis, rotation matrix, quaternion representation, and/or the like). As used herein, and for a device with a kinematic series, such as with a repositionable structure with a plurality of links coupled by one or more joints, the term “proximal” refers to a direction toward a base of the kinematic series, and “distal” refers to a direction away from the base along the kinematic series.

As used herein, the term “pose” refers to the multi-degree of freedom (DOF) spatial position and orientation of a coordinate system of interest attached to a rigid body. In general, a pose includes a pose variable for each of the DOFs in the pose. For example, a full 6-DOF pose for a rigid body in three-dimensional space would include 6 pose variables corresponding to the 3 positional DOFs (e.g., x, y, and z) and the 3 orientational DOFs (e.g., roll, pitch, and yaw). A 3-DOF position only pose would include only pose variables for the 3 positional DOFs. Similarly, a 3-DOF orientation only pose would include only pose variables for the 3 rotational DOFs. Further, a velocity of the pose captures the change in pose over time (e.g., a first derivative of the pose). For a full 6-DOF pose of a rigid body in three-dimensional space, the velocity would include 3 translational velocities and 3 rotational velocities. Poses with other numbers of DOFs would have a corresponding number of velocities translational and/or rotational velocities.

Aspects of this disclosure are described in reference to computer-assisted systems, which can include devices that are teleoperated, externally manipulated, autonomous, semiautonomous, and/or the like. Further, aspects of this disclosure are described in terms of an implementation using a teleoperated surgical system, such as the da Vinci® Surgical System commercialized by Intuitive Surgical, Inc. of Sunnyvale, California. Knowledgeable persons will understand, however, that inventive aspects disclosed herein may be embodied and implemented in various ways, including teleoperated and non-teleoperated, and medical and non-medical embodiments and implementations. Implementations on da Vinci® Surgical Systems are merely exemplary and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperated systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (with or without return to a human or animal anatomy) and for procedures on human or animal cadavers. Further, these techniques can also be used for medical treatment or diagnosis procedures that include, or do not include, surgical aspects.

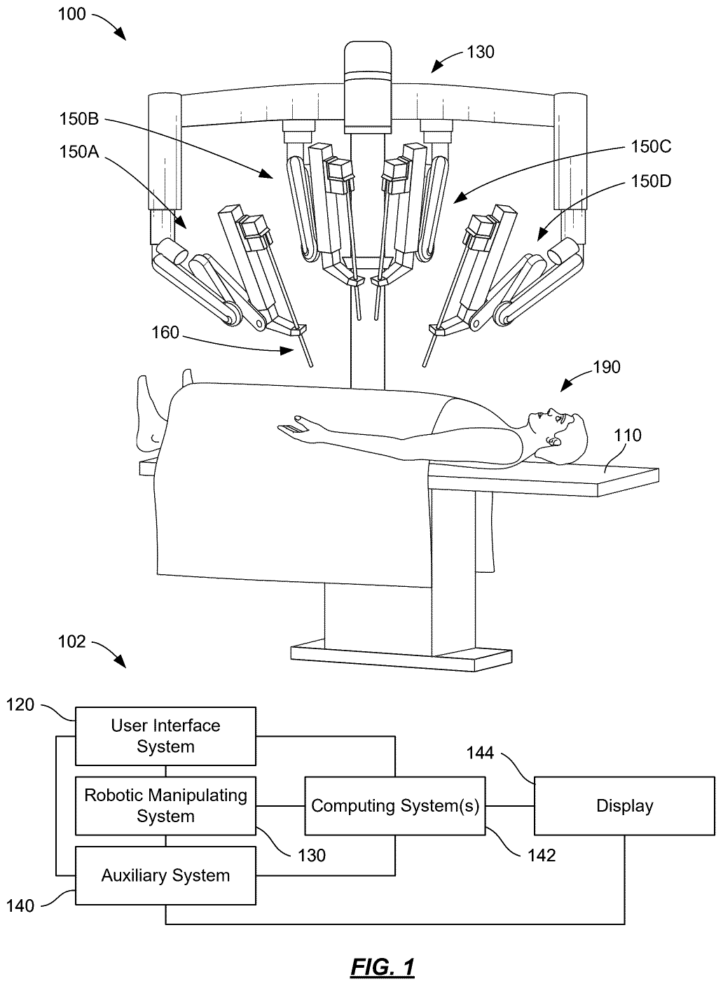

FIG. 1 shows an example computer-assisted system 100, in accordance with one or more embodiments.

In the example, a diagnostic or therapeutic medical procedure is performed on a patient 190 on an operating table 110. The computer-assisted system 100 may include a manipulator assembly 130 (e.g., a patient-side robotic device in a medical example). The manipulator assembly 130 may include at least one manipulator arm 150 (e.g., a robotic manipulator arm). A manipulator arm 150 may be any type of manipulator (e.g., a general-purpose robotic arm, a robotic arm designed for a specific application (e.g., a medical device robotic arm)). A manipulator arm 150 may include any number of links that are coupled by joints of any type (e.g., revolute joints, prismatic joints). One or more of the manipulator arms 150 may support a removably coupled instrument 160 (also called tool 160). In the manipulator assembly 130 and manipulator arm(s) 150 may maneuver the instrument 160 to a workspace through an entry location (e.g., enter the body of the patient 190 through a natural orifice such as the throat or anus, or through an Incision), while an operator (not shown) views the workspace (e.g., a surgical site in the surgical scenario) through a user interface system 120.

An image of the workspace may be obtained by an instrument 160 comprising an imaging device (e.g., an endoscope, an optical camera, an ultrasonic probe, etc. in a medical example). The imaging device can be used for imaging the workspace, and may be manipulated by one of the manipulator arms 150A-D of the manipulator assembly 130 so as to position and orient the imaging device. The auxiliary system 140 may process the captured images in a variety of ways prior to any subsequent display. For example, the auxiliary system 140 may overlay the captured images with a virtual control interface prior to displaying the combined images to the operator via the user interface system 120 or other display systems located locally or remotely from the procedure. One or more separate displays 144 may also be coupled with a control system 142 and/or the auxiliary system 140 for local and/or remote display of images, such as images of the procedure site, or other related images.

The number of instruments 160 used at one time generally depends on the task and space constraints, among other factors. If it is appropriate to change, clean, inspect, or reload one or more of the instruments 160 being used during a procedure, an assistant (not shown) may remove the instrument 160 from a manipulator arm 150, and replace it with the same instrument 160 or another instrument 160.

The computer-assisted system 100 may include a control system 142 (e.g., a computing system). The control system 142 may be used to process input provided by the user interface system 120 from an operator, such as to control the computer-assisted system 100. The control system 142 may also be used to process signals from other devices, from sensors, from any networks to which the control system 142 connects, etc. Example sensors include those associated with actuators or joints of the computer-assisted system, such as motor encoders, rotary or linear joint encoders, torque sensors, current sensors, accelerometers, force sensors, inertial measurement units, optical or ultrasonic sensors or imagers, RF sensors, etc. The control system 142 may further be used to provide an output (e.g., a video image for display by the display 144). The control system 142 may further be used to control the robotic manipulator assembly 130.

The control system 142 may include one or more computer processors, non-persistent storage (e.g., volatile memory, such as random access memory (RAM), cache memory), persistent storage (e.g., a hard disk, an optical drive such as a compact disk (CD) drive or digital versatile disk (DVD) drive, a flash memory, etc.), a communication interface (e.g., Bluetooth interface, infrared interface, network interface, optical interface, etc.), and numerous other elements and functionalities.

A computer processor of the control system 142 may be part or all of an integrated circuit for processing instructions. For example, the computer processor may be one or more cores or micro-cores of a processor. The control system 142 may also communicate with one or more input devices, such as a touchscreen, keyboard, mouse, microphone, touchpad, electronic pen, or any other type of input device.

A communication interface of the control system 142 may include an integrated circuit for connecting the control system 142 to a network (not shown) (e.g., a local area network (LAN), a wide area network (WAN) such as the Internet, mobile network, or any other type of network) and/or to another device, such as another control system 142.

Further, the control system 142 may communicate with one or more output devices, such as a display device (e.g., a liquid crystal display (LCD), a plasma display, touchscreen, organic LED display (OLED), projector, or other display device), a printer, a speaker, external storage, or any other output device. One or more of the output devices may be the same or different from the input device(s). Many different types of control systems exist, and the aforementioned input and output device(s) may take other forms.

Software instructions in the form of computer readable program code to perform embodiments of the disclosure may be stored, in whole or in part, temporarily or permanently, on a non-transitory computer readable medium such as a CD, DVD, storage device, a diskette, a tape, flash memory, physical memory, or any other computer readable storage medium. Specifically, the software instructions may correspond to computer readable program code that, when executed by a processor(s), is configured to perform one or more embodiments of the invention.

A control system 142 may be connected to or be a part of a network. The network may include multiple nodes. Each node may correspond to a computing system, or a group of nodes. By way of an example, embodiments of the disclosure may be implemented on a node of a distributed system that is connected to other nodes. By way of another example, embodiments of the invention may be implemented on a distributed computing system having multiple nodes, where each portion of the disclosure may be located on a different node within the distributed computing system. Further, one or more elements of the aforementioned computing system may be located at a remote location and connected to the other elements over a network.

While FIG. 1 shows the computer-assisted system 100 as a medical system, the following description is applicable to other scenarios and systems (e.g., medical scenarios or systems that are non-surgical, non-medical scenarios or computer-assisted systems, etc.).

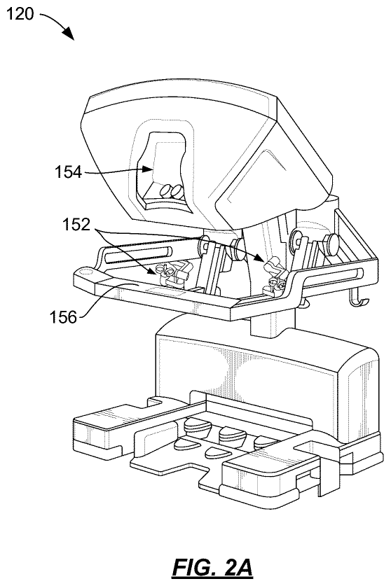

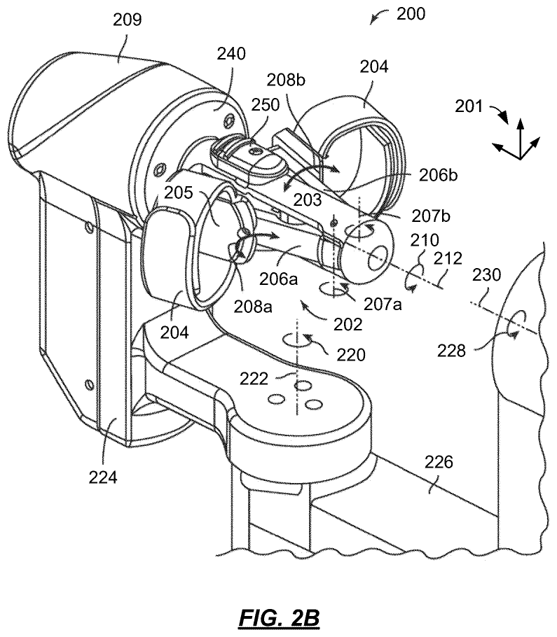

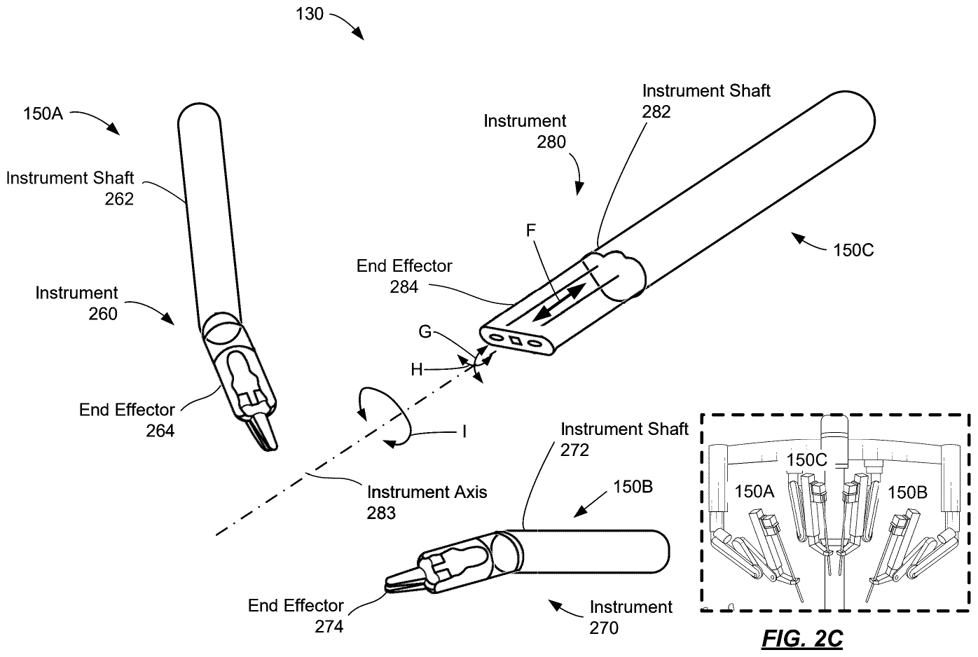

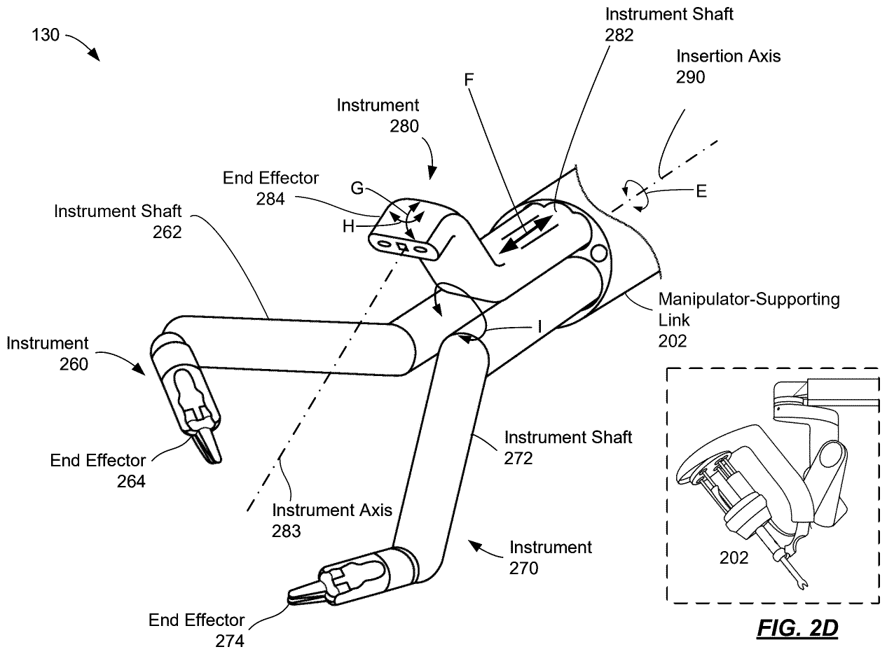

FIG. 2A shows a user interface system, in accordance with one or more embodiments.

In some embodiments, the user interface system 120 includes one or more input devices 152 operated by the operator (not shown). The one or more input devices 152 are contacted and manipulated by the hands of the operator, with one input device for each hand. Examples of such hand-input-devices include any type of device manually operable by human operator (e.g., joysticks, trackballs, button clusters, and/or other types of haptic devices typically equipped with multiple degrees of freedom). A more detailed description of an input device 152 is provided below in reference to FIG. 2B. Additionally, in some embodiments, position, force, and/or tactile feedback devices (not shown) are employed to transmit position, force, and/or tactile sensations from the instruments back to the operator's hands through the input devices 152.

The input devices 152 are supported by user interface system 120 and are shown as mechanically grounded, and in other implementations may be mechanically ungrounded. An ergonomic support 156 is provided in some implementations. For example, FIG. 2A shows an ergonomic support 156 including forearm rests on which the operator may rest his or her forearms while manipulating the input devices 152. In some examples, the operator performs tasks at a work site near the manipulator assembly 130 during a medical procedure by controlling the manipulator assembly 130 using the input devices 152.

A display unit 154 is included in the user interface system 120. The display unit 154 displays images for viewing by the operator. The display unit 154 provides the operator with a view of the workspace with which the manipulator assembly 130 interacts. The view can include, for example, stereoscopic images or three-dimensional images to provide a depth perception of the workspace and the instrument(s) 160 of the manipulator assembly 130 in the workspace. The display unit 154 can be moved in various degrees of freedom to accommodate the operator's viewing position and/or to provide control functions. Where a display unit (such as the display unit 154 is also used to provide control functions, such as to command the manipulator assembly, the display unit also includes an input device (e.g., another input device 152).

When using user interface system 120, the operator can sit in a chair or other support in front of user interface system 120, position his or her eyes to see images displayed by the display unit 154, grasp and manipulate the input devices 152, and rest his or her forearms on the ergonomic support 156 as desired. In some implementations, the operator can stand at the workstation or assume other poses, and the display unit 154 and input devices 152 may differ in construction and/or be adjusted in position (e.g., height, depth, etc.).

FIG. 2B is a perspective view of a controller portion 200 of an example input device 152 of the user interface system 120 shown in FIG. 2A.

In some implementations, the controller portion 200 includes one or more gimbal mechanisms. In the example of FIG. 2B, the controller portion 200 includes a handle 202 which is contacted by an operator to manipulate the input device. Further, in this example, the handle 202 includes two grips that each include a finger loop 204 and a grip member (depicted as grip members 206a, 206b). The two grip members 206a,b are positioned on opposite sides of a central portion 203 of the handle 202, and the grip members 206a,b can be grasped, held, or otherwise contacted by an operator's fingers. Each finger loop 204 is attached to a respective grip member 206a or 206b and can be used to secure an operator's fingers to the associated grip member 206a or 206b. In this example, finger contacts 205 can be connected or formed at the unconnected end of the grip members 206a,b to provide surfaces to contact the operator's fingers. The operator may also contact other portions of handle 202 while grasping the grip members 206a,b.

Each grip member 206a,b and finger loop 204 can be moved in an associated

degree of freedom (depicted as degrees of freedom 208a,b). In some examples, the grip members 206a,b are each coupled to the central portion 203 of the handle 202 at respective rotational couplings, allowing rotational movement of the grip members 206a,b about associated grip axes 207a,b, with respect to the central portion 203. As such, each grip member 206 can be moved in an associated degree of freedom (i.e., degree of freedom 208a about grip axis 207a and degree of freedom 208b about grip axis 207b) by an operator contacting the grip members 206a,b. In various implementations, a single grip member 206 (e.g., 206a) and finger loop 204 can be provided, or only one of the grip members 206 can be moved in the corresponding degree of freedom 208 while the other grip member (e.g., 206b) can be fixed with reference to the handle 202. For example, the positions of grip members 206a,b in their degrees of freedom 208a,b can control corresponding rotational positions of an instrument 160 or component thereof.

One or more grip sensors (not shown) can be coupled to the handle 202 and/or other components of the controller portion 200 and can detect the positions of the grip members 206a,b in their respective degrees of freedom 208a,b. The grip sensors can send signals describing sensed positions and/or motions to the control of control system 142 of the computer-assisted system 100. In some modes or implementations, the control system 142 can provide control signals to a device manipulated by the computer-assisted system 100 (e.g., manipulator assembly 130). For example, the positions of the grip members 206a,b in their respective degrees of freedom 208a,b can be used to control any of various degrees of freedom of an instrument 160 (or, in some instances, the distal end of an instrument 160) supported by the manipulator assembly 130.

Various implementations of an input device, such as the that depicted in the controller portion 200 of FIG. 2B, can provide one or more active actuators (e.g., motors, voice coils, etc.) to output active forces on the grip members 206a,b in the degrees of freedom 208a,b. For example, a sensor and/or actuator can be housed in central portion 203 or in housing 209 and coupled to the grip members 206a,b by a transmission. Some implementations can provide one or more passive actuators (e.g., brakes) or springs between the grip members 206a,b and the central portion 203 of the handle 202 to provide resistance in particular directions of the grips (e.g., movement in directions toward each other in degrees of freedom 208a,b).

The handle 202 can additionally be provided with a rotational degree of freedom 210 about a roll axis 212 defined between a first end and a second end of the handle 202. The roll axis 212 is a longitudinal axis in this example that extends approximately along the center of the central portion 203 of handle 202. The handle 202 can be rotated about the roll axis 212 with respect to a base member of the controller portion 200, such as a base member that includes the housing 209. For example, an operator can rotate the grip members 206a,b and central portion 203 as a single unit around the roll axis 212), with respect to the housing 209, to provide control of manipulator assembly 130 or other elements.

Additionally, one or more input sensors (not shown) can be coupled to the handle 202 to detect the orientation of the handle 202 in the rotational degree of freedom 210. For example, the sensor can send signals describing the orientation to the control system 142 that can provide control signals to the manipulator assembly 130 as described above. For example, rotation of the handle 202 in the rotational degree of freedom 210 can control a particular degree of freedom of an instrument 160 of the manipulator assembly 130 that is different than another degree of freedom controlled by the degrees of freedom 208a,b of the grip members 206a,b.

Some implementations of the controller portion 200 can provide one or more actuators to output forces on the handle 202 (including grip members 206a,b and finger loops 204 in the rotational degree of freedom 210. For example, a sensor and/or actuator can be housed in the housing 209 and coupled to the handle 202 by a shaft extending through the central portion 203 of the handle 202.

In various implementations, the handle 202 can be provided with additional degrees of freedom. For example, a rotational degree of freedom 220 about a yaw axis 222 can be provided to the handle 202 at a rotational coupling between an elbow shaped link 224 and a link 226, where the elbow shaped link 224 is coupled to the handle 202 (e.g., at the housing 209). In this example, the yaw axis 222 intersects and is orthogonal to the roll axis 212. Additional degrees of freedom can similarly be provided. For example, the link 226 can be elbow-shaped and a rotational coupling can be provided between the other end of link 226 and another link (not shown). A rotational degree of freedom 228 about an axis 230 can be provided to the handle 202 at the rotational coupling. In some examples, the controller portion 200 can allow movement of the handle 202 within the workspace of the user interface system 120 with a plurality of degrees of freedom (e.g., six degrees of freedom including three rotational degrees of freedom and three translational degrees of freedom). One or more additional degrees of freedom can be sensed by associated input sensors and/or actuated by actuators (e.g., motors, etc.), similarly as described above for the degrees of freedom, coupled to the controller portion 200. In various implementations, sensors can sense positions of the handle in a degree of freedom, or sense orientations of the handle in a degree of freedom, or sense positions and orientations of the handle in multiple degrees of freedom. For example, positions in a translational degree of freedom and orientations in a rotational degree of freedom can be sensed by one or more associated input sensors. In some examples, a position in a translational degree of freedom and/or orientation in a rotational degree of freedom can be derived from rotations of components (e.g., links of a linkage) coupled to the handle 202 as sensed by rotational sensors. Some implementations can include linear sensors that can directly sense translational motion of one or more components coupled to the handle 202. In some implementations, each additional degree of freedom of the handle 202 can control a different degree of freedom (or other motion) in the manipulator assembly 130.

In an example implementation, the handle 202 is mechanically grounded, i.e., supported in space by a kinematic chain with an end stationary at mechanical ground, such as a floor, wall, or ceiling. For example, the housing 209 can be coupled to a mechanical linkage that is coupled to the ground or an object connected to ground, providing a stable platform for the use of the controller portion 200. For example, a grounded mechanical linkage can be connected to a base member (e.g., with one or more rotary couplings, ball joints, or other couplings, including linear joints). The mechanical linkage can provide six or more degrees of freedom to the handle 202.

In the example of FIG. 2B, the handle 202 includes one or more control switches 240. As depicted, the one or more control switches 240 can be coupled to the central portion 203 or to mechanisms within central portion 203. For example, two control switches 240 can be positioned on opposite sides of axis 212, and/or additional control switches can be provided. In some examples, a control switch 240 has a portion that can slide parallel to the axis 212 (e.g., as directed by an operator's finger) or the control switch portion can be depressed. In some implementations, the control switch 240 can be moved to various positions to provide particular command signals (e.g., to select functions, options, or modes of the user interface system 120 and/or input device). In some implementations, one or more of the control switches 240 can be implemented as a button (e.g., depressed in a direction, such as perpendicular to the axis 212 or other direction), a rotary dial, a switch that moves perpendicular to the axis 212, or other type of input control. Control switches 240 can use electromagnetic sensors, mechanical switches, magnetic sensors, or other types of sensors to detect positions of the switch.

As previously stated, the computer-assisted system 100 can be a teleoperated system in which the user interface system 120 is, or is included in, a “leader” device that controls at least a portion of the manipulator assembly 130 (which in literature describing teleoperated systems may be known as a “follower” device).

In general, a control system 142 (e.g., a computing system) receives control signals from the user interface system 120 and generates actuation signals which are sent to manipulator assembly 130. The control system 142 can also receive sensor signals that indicate positions, orientations, states, and/or changes of various follower components (e.g., manipulator arm elements) from the manipulator assembly 130 and send actuation signals to the user interface system 120 to provide force, torque, and/or position feedback to the operator.

In one or more embodiments, a user interface system 120 is equipped with two controller portions 200 (one for each of the operator's hands) to control elements of the manipulator assembly 130 (e.g., instruments 160A-D attached to different manipulator arms 150A-D). An input device coordinate frame 201 is associated with the controller portions 200. In other words, the configuration (position and/or orientation) of the input device(s) 152 are determined within the input device coordinate frame 201 such that operations (e.g., kinematic operations) may be performed relative to a base frame of the manipulator assembly 130. For example, the position and/or orientation of an instrument 160 may be determined in the base frame using forward kinematics of the manipulator assembly 130 based on the configuration of one or more input devices 152.

While FIGS. 1-2B show various configurations of components, other configurations may be used without departing from the scope of the disclosure. For example, various components may be combined to create a single component. As another example, the functionality performed by a single component may be performed by two or more components. While examples of particular manipulator assemblies, particular repositionable structures, instruments, input systems, and controller portions are shown, the disclosure generalizes to any type of manipulator assemblies, repositionable structures, instruments, input systems, and controller portions with any number of degrees of freedom. Further, while components are often described in context of medical scenarios such as surgical scenarios, embodiments of the disclosure are equally applicable to other domains that involve robotic manipulation, e.g., non-surgical scenarios or systems, non-medical scenarios or systems, and/or the like.

FIGS. 2C-2D show examples of a manipulator assembly 130, in accordance with one or more embodiments.

In some embodiments, the manipulator assembly 130 introduces a set of instruments 260, 270, 280 (e.g., contained within one or more cannula) to a work site through an aperture (e.g., a port, an entry guide, an orifice). The manipulator assembly 130 includes a manipulator-supporting link 250 that may be rotated (arrow E) about insertion axis 290 of the manipulator-supporting link 250. The three instruments 260, 270, and 280 are supported by the manipulator-supporting link 250. Translational movement of each of the instruments 260, 270, 280 may be independently controlled (e.g., arrow F, for simplicity shows translation along the insertion axis for instrument 280). Each of the instruments 260, 270, 280 include an end effector 264, 274, 284 that may be positioned with one or more degrees of freedom (e.g., a 6 DOF instrument) by controlling the position and orientation of the joints and the links of instrument shafts 262, 272, 282 and, respectively.

Instruments 260 and 270 are illustrated as equipped with end effectors 264, 274 that each include one or more degrees of freedom. For example, each end effector may be able to translate position in three dimensions or rotate orientation (e.g., pitch, yaw, roll). In case of a jawed end effector, an additional degree of freedom may further enable and open/close operation of the jaws. As shown, instruments 260 and 270 may be two independently teleoperated instruments associated with a separate input device 152 (e.g., one left hand input device 152L for the left instrument 270 and one right hand input device 152R for the right instrument 260).

Instrument 280 is illustrated as equipped with an imaging device (e.g., an endoscopic camera) as the end effector 284. The imaging device may be any type of imaging sensor. The imaging device may be a general-purpose monoscopic or stereoscopic camera or it may be a specialized instrument (e.g., an endoscope). Additional details may be found in U.S. Pat. No. 8,620,473, (e.g., in FIG. 8 showing an articulable imaging system (1750) and the associated description).

The imaging device may be movable with one or more degrees of freedom. The imaging device may be able to articulate separately from other instruments 260, 270 that may be present in the workspace. The imaging device may have its own joints, in addition to the joints of the manipulator assembly 130. Any number of joints may be present, as long as the number of joints is sufficient to enable movement. More generally, the combination of the manipulator assembly 130 and the imaging device is not limited to a particular kinematic configuration.

For example, the end effector 284 may be able to change orientation in pitch and yaw degrees of freedom (arrows G and H, respectively). While FIG. 2C shows a jointed configuration of instrument shaft 282 that offsets the end effector 284 from instrument axis 283, the end effector 284 may be aligned with the instrument axis 283 (e.g., the imaging device is aligned with the instrument axis 283). In some embodiments, instrument 280 further includes a degree of freedom for rotation of the instrument shaft 282 (arrow I). Thus, imaging device of instrument 280 may be used to observe operations performed using the end effectors 264, 274 of instruments 260, 270 by an operator viewing images captured from the end effector 284. If the operator wants to change the field of view of the imaging device, for example to view the operations performed using the end effectors 264, 274 from a different angle, the instrument 280 may be translated and/or rotated by control of instrument 280 (e.g., by an input device 152).

While the manipulator assembly 130 shown in FIG. 2C includes a manipulator-supporting link 250 with a plurality of instruments 260, 270, 280, other embodiments are also possible. For example, FIG. 2D shows each instrument may be independently supported (e.g., supported by separate manipulator arm 150A-C shown in FIG. 1).

As discussed above, any movement of the imaging device will alter the spatial relationship between the field of view and instruments in the workspace. Because the coordination of the operator (e.g., manipulating input device(s) 152) in the input device coordinate frame 201) relies on the operator's observation of the field of view, a mismatch (e.g., caused by a roll movement of the imaging device), would be undesirable.

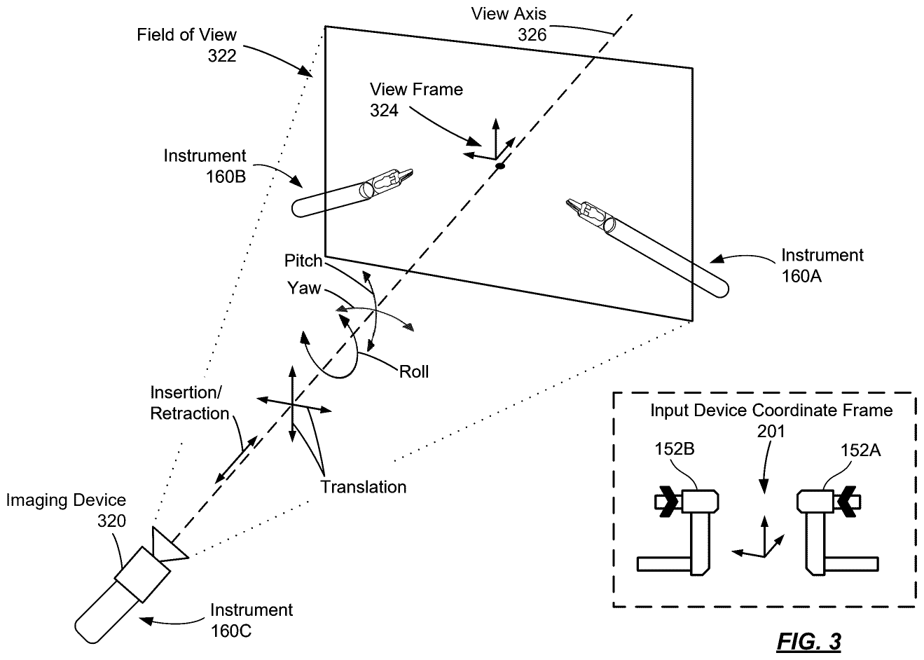

FIG. 3 is a simplified diagram showing elements of a manipulator assembly 130 in a workspace, in accordance with one or more embodiments.

In FIG. 3, the manipulator assembly 130 includes the instrument 160C equipped with an imaging device 320, the first instrument 160A, and the second instrument 160B. When operating the computer-assisted system 100 to perform a procedure, the operator may rely, at least partially, on visual feedback from the workspace. The workspace may include a target site, and the operator may, for example, want to visually inspect the target site and/or interact with the target site (e.g., by performing a procedure). The imaging device 320 may provide visual feedback based on a field of view 322 of the imaging device 320 (e.g., a 3D or 2D view of the field of view 322 determined by the optical characteristics of the imaging device 320).

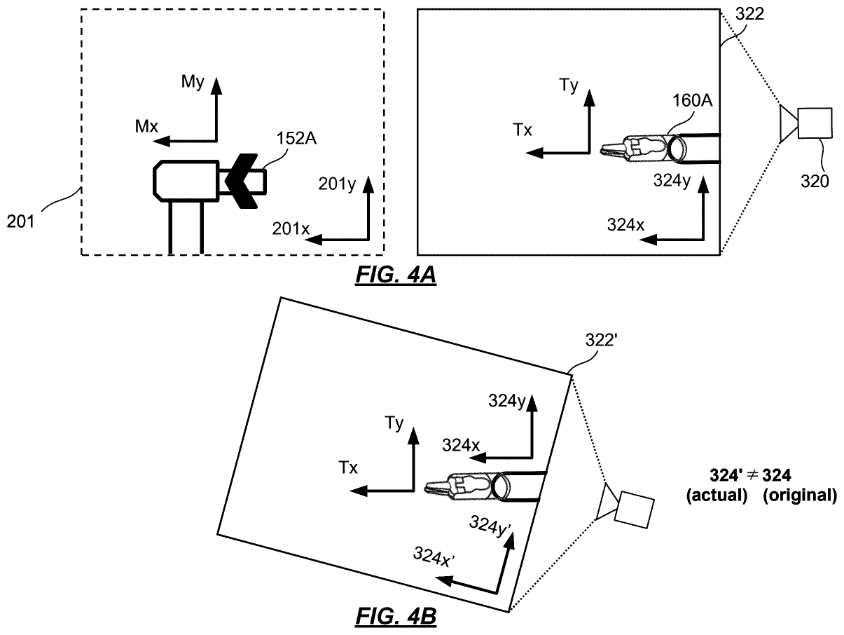

A view coordinate frame 324 is associated with the field of view 322. The view coordinate frame 324 is fixed relative to the field of view 322 (e.g., at the center of the field of view 322 as shown in FIG. 3, at a corner of the field of view as shown in FIGS. 4A-B). The view coordinate frame 324 is defined by a plurality of view frame vectors (e.g., orthogonal unit vectors, any appropriate coordinate vector system). In some embodiments, a first view frame vector defines a horizontal axis of the field of view 322, a second view frame vector defines a vertical axis of the field of view 322 and a third view frame vector is parallel to a view axis 326 that extends perpendicular to the field of view 322. It will be appreciated that other configurations of view frame vectors may be used to define the view coordinate frame 324.

The operator may want to change the field of view 322 for various reasons (e.g., to better observe the target site). While the field of view 322 may be modifiable using optical and digital operations (e.g., optical/digital zoom, digital translation and/or roll operations), in the following discussion, the field of view 322 is assumed to be changed by movement of the imaging device 320. The position and/or orientation of the imaging device 320 may be updated as desired by the operator, by changing the configuration of the joints of the manipulator assembly 130 (e.g., movement of manipulator assembly 130, manipulator arm 150, and/or instrument 160C).

In some embodiments, one or more of the operating modes of the manipulator assembly 130 may enable the operator to control movement of the field of view 322 by moving an input device 152 (e.g., handle 202 of the controller portion 200 of the input device 152A or 152B) which translates into commanded motion of the imaging device 320. Examples of robotic systems operating in different operating modes when receiving user inputs are provided in U.S. Pat. No. 9,586,323. U.S. Pat. No. 9,586,323 is hereby incorporated by reference in its entirety. The motion of the imaging device 320 may include one or more components (e.g., insertion/retraction/translation components, rotation components (e.g., a roll component, a pitch component, a yaw component)), as illustrated in FIG. 3.

In some embodiments, the configuration (position and/or orientation) of the imaging device 320 and therefore the field of view 322 may be directly manipulated by the operator (e.g., manual adjustment of imaging device 320, instrument 160C, manipulator arm 150, manipulator assembly 130).

In general, the movement of the imaging device 320 and/or the field of view 322 may be summarized with a roll component and one or more non-roll components comprising the one or more translation components, the pitch component, the yaw component, and/or the insertion/retraction component. The motion of the field of view 322 may be described relative to any reference frame. In some embodiments, the motion of the field of view 322 may be described relative to the view coordinate frame 324 (e.g., a current view coordinate frame may be obtained relative to a previous view coordinate frame, or a future view coordinate frame may be obtained relative to a current view coordinate frame). In some embodiments, the motion of the field of view 322 may be described relative to a base of the manipulator assembly 130, which may be stationary or may be movable (e.g., installed on tracks enabling a linear motion, installed on another manipulator arm).

In FIG. 3, the instruments 160A, 160 Beach support end effectors within the workspace being imaged by imaging device 320. In some embodiments, during a teleoperation control session, the movements of the instruments 160A, 1650B mimic the operator's manipulation of the input devices 152A, 152B, respectively, in the input device coordinate frame 201 (e.g., within the user interface system 120 of FIG. 2A). In this non-limiting example, the instrument 160A is manipulated based on input from input device 152A and the instrument 160B is manipulated based on input from input device 152B. It will be appreciated that the manipulator assembly 130 may be configured with any association between one or more input device(s) 152 and instrument(s) 160 in the workspace. For example, in some embodiments, control of any instrument(s) 160 may be switched between different input device(s) 152 or vice versa.

A high degree of coordination between a given input device 152 and the manipulator assembly 130 is required, especially during teleoperation control session (as known as a “following” mode) where the movement of an associated instrument 160 mimics the movement of the input device 152. If coordination between the movements of the input device 152 in the input device coordinate frame 201 the movements of the manipulator assembly 130 in the workspace is lost or degraded, operation of the computer-assisted system 100 may become non-intuitive to the operator. For example, lack of coordination may result in unintended movements of the associated instrument 160 (e.g., moving in the wrong direction). Therefore, one or more embodiments disclosed herein are directed to identifying and rectifying configurations of the computer-assisted system 100 that may result in non-intuitive control schemes and/or non-intuitive motion.

As discussed above, in some embodiments, an imaging device 320 of the manipulator assembly 130 may be configured to move such that the operator can view the workspace from different perspectives. However, any movement of the imaging device 320 will alter the spatial relationship between the field of view 322 of the workspace and the input device coordinate frame 201. In other words, while the mapping between a given input device 152 and the associated instrument 160 may remain unchanged within the teleoperation control session, the field of view 322 that the operator views in the display unit 154 may be offset (e.g., rotated) causing the perceived motion of the associated instrument 160 to not match the actual motion of the operators hands in the given input device 152 (e.g., the associated instrument 160 moves at a different perceived angle compared to the operator's command).

In some instances, the offset of the imaging device 320 may be caused by direct manipulation (e.g., manual adjustment) or indirect manipulation (e.g., drift) of the imaging device 320, the instrument 160C, the manipulator arm 150, and/or the manipulator assembly 130. Because the manipulation is not prompted, or accounted for, by the control system 142, subsequent movement of the manipulator assembly 130 will become non-intuitive when viewed through the imaging device 320.

In some instances, the offset of the imaging device 320 may be caused if an actual movement of the imaging device 320 is imperfect or otherwise different from a commanded movement (e.g., obstructed movement, back driving of a positioning motor). As a result of the imperfect movement, the new field of view 322 may not match the expected spatial relationship and subsequent movement of the manipulator assembly 130 will become non-intuitive when viewed through the imaging device 320.

In some embodiments, the control system 142 monitors the roll orientation of the imaging device 320 to identify a configuration that may cause unintended motion. For example, the control system 142 may be configured to record a first roll orientation of the imaging device when a first teleoperation control session is enabled. The control system may subsequently determine whether or not a roll difference between the first roll orientation and a current roll orientation exceeds a threshold. When the threshold is exceeded, the control system may terminate the first teleoperation control session to prevent a non-intuitive control scheme or unintended motion.

FIGS. 4A-4B show example fields of view of an imaging device, in accordance with one or more embodiments.

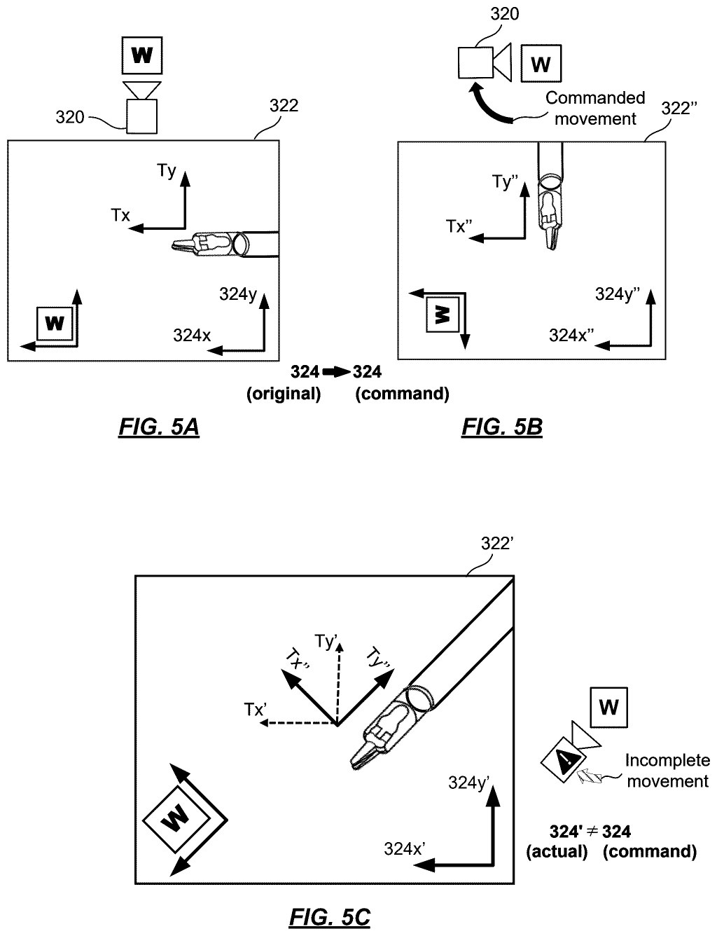

In the FIGS. 4A-5C, a first instrument 160A is manipulated based on input from a first input device 152A. Based on this association between the first input device 152A and the first instrument 160A, a transformation is determined between the input device coordinate frame 201 and a workspace coordinate frame W (not shown). The transformation may include one or more parameters (e.g., scalar or vector quantities) and/or one or more equations (e.g., coordinate transformation equations, matrices) to relate the two coordinate frames and characterize the synchronization between the first input device 152A with the manipulator assembly 130 (e.g., the first instrument 160A supported by the manipulator assembly 130).

In some embodiments, a scaling factor may be a parameter of the transformation. The scaling factor may be a scalar value that converts the magnitude of movement vector M to a different magnitude of in the workspace coordinate frame W. The scaling factor may be manually or automatically controlled or may be fixed (e.g., based on a type of procedure).

In some embodiments, a gain setting, or sensitivity setting, may be a parameter of the transformation. The gain setting may be one or more scalar or vector quantities that affect the responsiveness of the first instrument 160A to movements of the first input device 152S. For example, the kinematics of the first input device 152A (e.g., velocity, acceleration, jerk) may be scaled by the gain setting to control the kinematics of the first instrument 160A within the workspace coordinate frame W.

The configuration (e.g., pose, position, orientation, kinematic configuration, etc.) of the imaging device 320 in the workspace coordinate frame W is an important element of correctly determining and applying any given transformation for a teleoperational control session.

In FIG. 4A, the first transformation is determined based on the current configuration of the imaging device 320. Therefore, the first transformation causes movements of the first instrument 160A in the view coordinate frame 324 (i.e., movement vector T with components Tx and Ty) to synchronize with the movements of the first input device 152A in the input device coordinate frame 201 (i.e., movement vector M with components Mx and My). In other words, because the transformation correctly accounts for the configuration of the imagine device 320 in the workspace coordinate frame W, the movement vector T in the workspace coordinate frame W is aligned with the view coordinate frame 324.

As viewed by the operator in the field of view 322, the perceived movement of the first instrument 160A is intuitive and mimics the motion of the first input device 152A in the input device coordinate frame 201. In other words, a horizontal movement Mx of the first input device 152A in the input device coordinate frame 201 causes the first instrument 160A to move in a horizontal direction Tx in the view coordinate frame 324; likewise, a vertical movement My of the first input device 152A in the input device coordinate frame 201 causes the first instrument 160A to move in a vertical direction Ty in the view coordinate frame 324.

In FIG. 4B, a roll movement of imaging device 320 along the view axis 326 creates a new view coordinate frame 324′ that is rotationally offset from the view coordinate frame 324 in FIG. 4A. However, if the transformation from FIG. 4A is not updated to account for the change in configuration of the imaging device 320 (i.e., the transformation is based on an outdated or incorrect configuration of the manipulator assembly 130), the movements of the first instrument 160A in the workspace coordinate frame W will no longer aligned with the new view coordinate frame 324′. Because the transformation from FIG. 4A is unchanged, movements of the first input device 152A cause the same movements of the first instrument 160A in the workspace coordinate frame W as in FIG. 4A. However, the new view coordinate frame 324′ does not have the same spatial relationship with the workspace coordinate frame W as the view coordinate frame 324 from FIG. 4A, and the perceived motion in the new view coordinate frame 324′ will have a rotational offset relative to the movements of the first input device 152A in the input device coordinate frame 201.

As viewed by the operator in the new field of view 322′, the perceived movement of the first instrument 160A is no longer intuitive due to the rotational offset from the motion of the first input device 152A in the input device coordinate frame 201. The horizontal movement Mx of the first input device 152A in the input device coordinate frame 201 still causes the first instrument 160A to move in the direction Tx, which is rotationally offset from the horizontal direction 324x′ in the new view coordinate frame 324′; likewise, the vertical movement My of the first input device 152A in the input device coordinate frame 201 still causes the first instrument 160A to move in the direction Ty, which is offset from the vertical direction 324y′ in the new view coordinate frame 324′.

In other words, as long as the transformation from FIG. 4A fails to account for the change in configuration of the imaging device 320, using the first input device 152A to control the first instrument 160A is less intuitive or non-intuitive because the operator must identify and account for the rotational offset between view coordinate frames 324, 324′.

While embodiments of this disclosure are described with respect to horizontal and vertical movements in the workspace coordinate frame W, it will be appreciated that any frame of reference for the movements may be used. For example, the coordinate spaces of the input device, field of view, and/or workspace do not need to be rectilinear. Any coordinate system may be used as long as the transformation includes the necessary information to relate one coordinate system to another.

FIGS. 5A-5C show example fields of view of an imaging device, in accordance with one or more embodiments.

FIGS. 5A-5C demonstrate another example of the importance of accounting for the actual configuration of the imaging device 320 in the workspace coordinate frame W.

In FIG. 5A, the first transformation synchronizes movements of the first instrument 160A in the view coordinate frame 324 with the movements of the first input device 152A in the input device coordinate frame 201 (not shown). In other words, similar to FIG. 4A, the first transformation correctly accounts for the configuration of the imagine device 320 relative to the workspace coordinate frame W such that the movement vector T in the workspace coordinate frame W is aligned with the view coordinate frame 324.

As viewed by the operator in the field of view 322, the perceived movement of the first instrument 160A is intuitive and mimics the motion of the first input device 152A in the input device coordinate frame 201. In other words, a horizontal movement Mx of the first input device 152A in the input device coordinate frame 201 causes the first instrument 160A to move in a horizontal direction Tx in the view coordinate frame 324, which is aligned with the horizontal direction 324x in the view coordinate frame 324; likewise, a vertical movement My of the first input device 152A in the input device coordinate frame 201 causes the first instrument 160A to move in a vertical direction Ty, which is aligned with the vertical direction 324y in the view coordinate frame 324.

In FIG. 5B, a movement command includes instructions that cause the imaging device 320 to rotate relative to the workspace coordinate frame W. Based on the configuration of the manipulator assembly 130 (e.g., how the joints and/or links support the imaging device 320) and the known kinematics of the commanded movement, a computer-assisted system 100 (e.g., the control system 142) can determine an expected transformation that accounts for the movement command.

In other words, a movement command may include the commanded movement for the imaging device 320 and instructions for updating the original transformation to the expected transformation. By updating the first transformation based on the commanded movement, synchronized motion of the first input device 152A in the input device coordinate frame 201 with motion of the first instrument 160A in the original field of view 322 can be continued into expected field of view 322″ (e.g., “following” motion can be continued without stopping or recalibrating the computer-assisted system 100).

In this non-limiting example, the movement command includes a 90 degree rotation about the view axis 326 of the imaging device 320. As viewed by the operator in the expected field of view 322″, the perceived movement of the first instrument 160A is intuitive and mimics the motion of the first input device 152A in the input device coordinate frame 201. In other words, a horizontal movement Mx of the first input device 152A in the input device coordinate frame 201 causes the first instrument 160A to move in a vertical direction in the workspace due to the movement command, which corresponds to a horizontal movement Tx″ aligned with the horizontal direction 324x″ in the expected view coordinate frame 324″; likewise, a vertical movement My of the first input device 152A in the input device coordinate frame 201 causes the first instrument 160A to move in a horizontal direction in the workspace due to the movement command, which corresponds to a vertical movement Ty″ aligned with the vertical direction 324y″ in the expected view coordinate frame 324″.

When the configuration of the manipulator assembly 130 is known with complete accuracy, a high degree of coordination between the first input device 152A and the first instrument 160A can be maintained by repeated updating to the expected transformation. However, if any configuration information is corrupted, lost, or otherwise incorrect, the expected new transformation will be incorrect and subsequent operation of the manipulator assembly 130 may result in unintended movements.

As shown in FIG. 5C, if the commanded movement of the imaging device is offset from the actual movement of the imaging instrument for any reason (e.g., obstructed by an object in the workspace, restricted by the manipulator assembly 130 (e.g., tension on a wire or cable, lack of power in an actuator/motor), overdriven by the manipulator assembly 130), the actual configuration of the imaging device 320 (i.e., the interrupted view coordinate frame 324′ with interrupted field of view 322′) will not match the expected configuration that is based on the commanded movement. Therefore, the expected transformation implemented with the move command will be incorrect for the interrupted field of view 322′.

As viewed by the operator in the interrupted field of view 322′, the perceived movement of the first instrument 160A is non-intuitive and no longer synchronizes with the motion of the first input device 152A with input device coordinate frame 201. In other words, under the expected transformation, a horizontal movement Mx of the first input device 152A in the input device coordinate frame 201 causes the first instrument 160A to move in a vertical direction in the workspace (as in FIG. 5B), which corresponds to a diagonal movement Tx″, which is offset from the horizontal direction 324x′ in the interrupted view coordinate frame 324′; likewise, a vertical movement My of the first input device 152A in the input device coordinate frame 201 causes the first instrument 160A to move in a horizontal direction in the workspace (as in FIG. 5B), which corresponds to a diagonal movement Ty″, which is offset from the vertical direction 324y′ in the interrupted view coordinate frame 324′.

To prevent unintended and/or non-intuitive motion as described above with respect to FIGS. 4B and 5C, one or more embodiments of the invention provide a method of maintaining/evaluating coordination of the computer-assisted system 100.

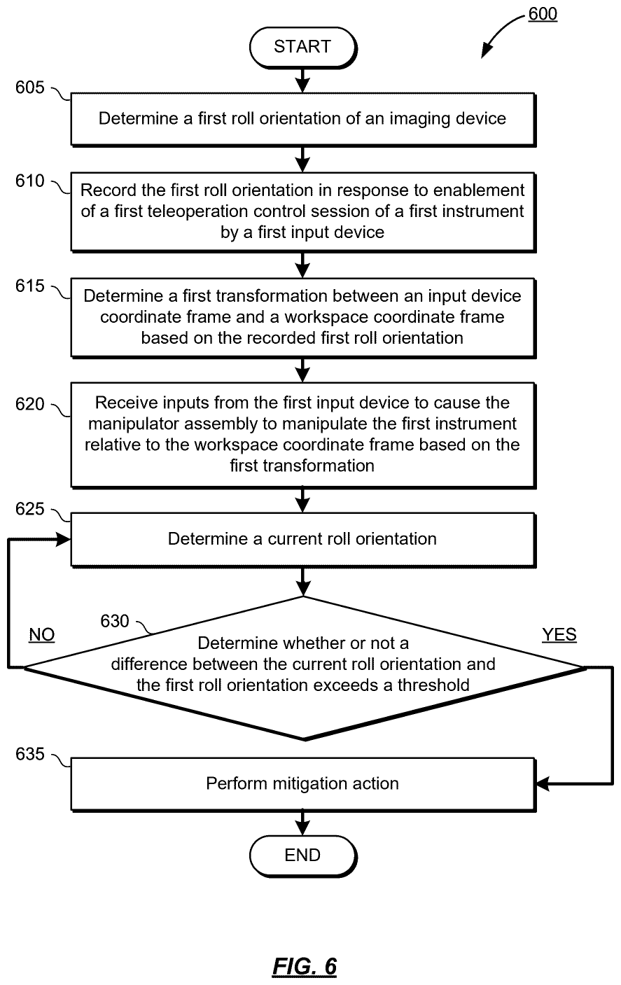

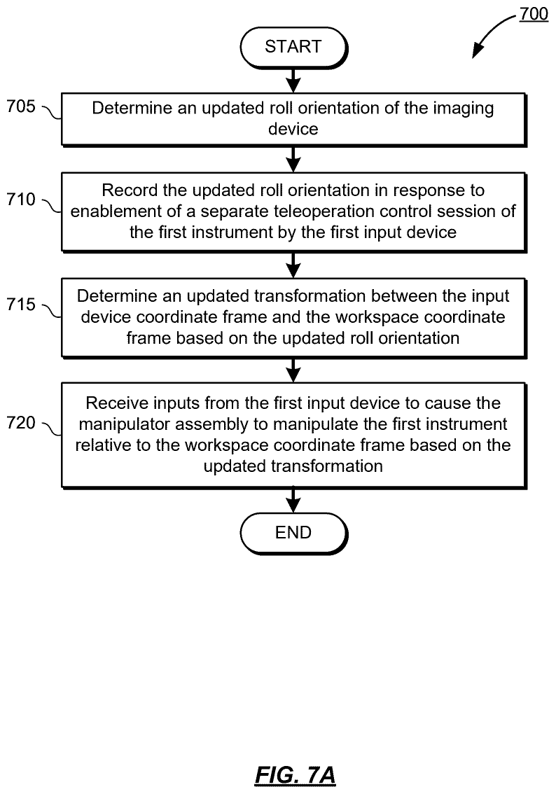

FIG. 6 shows a method according to one or more embodiments.

At 605, the control system 142 determines a first roll orientation of the imaging device 320. The first roll orientation of the imaging device 320 may be determined based on one or more sensors (e.g., as motor encoders, rotary or linear joint encoders) of the manipulator assembly 130 that are associated with actuators and/or joints that support the imaging device 320. The control system 142 may determine the roll orientation directly from a signal or information from the one or more sensors or may calculate the roll orientation based on configuration information of the manipulator assembly 130 (e.g., Denavit-Hartenberg (DH) parameters).

In some embodiments, a roll orientation of the imaging device is obtained from an encoder in a portion of the manipulator assembly 130 that supports the imaging device 320. The roll orientation may be obtained by the control system 142 or by a processor that is specific to a portion of the manipulator assembly 130 that supports the imaging device (e.g., the processing kernel of a manipulator arm 150 that supports the imaging device 320).

In some embodiments, the control system 142 enables a teleoperational control session based on a matching grip configuration of an input device 152 (e.g., first input device 152A) and an associated instrument 160 (e.g., first instrument 160A) (e.g., the configuration of the associated instrument 160 and/or an end effector). In some embodiments, the roll orientation (e.g., the first roll orientation) of the imaging device 320 is determined at the time of or during the process of enabling a teleoperational session. Furthermore, in some embodiments, the roll orientation of the imaging device 320 may be determined during an operation to match the orientation of the input device 152 in the input device coordinate frame 201 to an orientation of the associated instrument 160 in the workspace coordinate frame W.

At 610, the control system 142 records the first roll orientation of the imaging device 320 in response to enablement of a first teleoperational control session of the first instrument 160A by the first input device 152A.

In some embodiments, the control system 142 enables the first teleoperational control session based on a matching grip configuration of the first input device 152A and the first instrument 160A (e.g., the configuration of the first instrument 160A and/or an end effector). Because the first roll orientation is an important element of correctly determining and applying a transformation to the first teleoperational control session, the first roll orientation of the imaging device 320 may be determined and/or recorded upon successfully matching the grip configuration.

At 615, the control system 142 determines a first transformation between the input device coordinate frame 201 and a workspace coordinate frame W based on the first roll orientation of the imaging device 320. As discussed above, the first transformation includes one or more parameters/equations that convert that a movement vector M in the input device coordinate frame 201 to a movement vector T in the workspace coordinate frame W that is aligned with the view coordinate frame 324 of the imaging device 320.

At 620, the control system 142 receives inputs from the first input device 152A to cause the manipulator assembly 130 to manipulate the first instrument 160A relative to the workspace coordinate frame W based on the first transformation.

At 625, the control system 142 determines a current roll orientation of the imaging device 320. Similar to 605, the current roll orientation of the imaging device 320 may be determined based on one or more sensors (e.g., as motor encoders, rotary or linear joint encoders) of the manipulator assembly 130 that are associated with actuators and/or joints that support the imaging device 320. The control system 142 may determine the current roll orientation directly from a signal or information from the one or more sensors or may calculate the current roll orientation based on configuration information (e.g., pose, position, orientation, kinematic configuration, etc.) of the manipulator assembly 130.

In some embodiments, the current roll orientation is obtained from an encoder in a portion of the manipulator assembly 130 that supports the imaging device 320.

In some embodiments, the current roll orientation may be obtained by a processing kernel of a manipulator arm 150 that supports the imaging device 320 (e.g., a dedicated processing system) that is supervised by another processing kernel of the control system 142 (e.g., a supervisor processing system, a processing kernel of another manipulator arm 150). In this configuration, the current roll orientation may be actively monitored in coordination with any movement or action of the manipulator arm 150 that is controlled by the processing kernel (e.g., at the highest refresh rate appropriate to the manipulator arm 150) during the first teleoperational control session. For example, the current roll orientation may be determined during every processing cycle of the processing kernel, every given number of processing cycles (e.g., every 5 cycles), or at any other rate. As discussed in further detail below with respect to FIG. 8, the current roll orientation may be monitored by multiple systems in parallel (e.g., corresponding to different teleoperational control sessions).

At 630, the control system 142 determines whether or not a difference between the current roll orientation and the first roll orientation exceeds a first threshold. When the determination is YES (i.e., the difference between the current roll orientation and the first roll orientation exceeds the first threshold), the method continues to 635. When the determination is NO (i.e., the difference between the current roll orientation and the first roll orientation is equal to or less than the first threshold), the method returns to 625 (e.g., immediately or after a delay).

As discussed above with respect to FIGS. 4A-5C, the difference between the current roll orientation and the first roll orientation may be caused by any motion of the imaging device 320 that is not accounted for by the control system 142 (e.g., manual movement by an operator, back driving of a motor/servo, an offset from a movement command).

In some embodiments, the control system 142 determines the difference between the current roll orientation and the first roll orientation.

In some embodiments, the difference may be determined by a processing kernel of a manipulator arm 150 that supports the imaging device 320 (e.g., a dedicated processing system) that is supervised by another processing kernel of the control system 142 (e.g., a supervisor processing system, a processing kernel of another manipulator arm 150). In this configuration, the current roll orientation may be actively monitored in coordination with any movement or action of the manipulator arm 150. As discussed in further detail below with respect to FIG. 8, the difference between the current roll orientation and the roll orientation corresponding to the enablement of a teleoperational control session may be monitored by multiple systems in parallel (e.g., corresponding to different teleoperational control sessions).

In some embodiments, to avoid unnecessary mitigation action due to noise in determination of the roll orientation, the first threshold may be set to a non-zero value. For example, the threshold may be an angular value greater than 10 degrees (e.g., 15 degrees, 20 degrees, 25 degrees, 30 degrees).

In some embodiments, the first threshold may be set to a non-zero value to avoid unnecessary use of computational resources (e.g., correcting for a roll orientation that is not perceptible to the operator).

In some embodiments, the first threshold is determined based on a scaling factor of the first transformation. For example, a higher scaling factor increases the magnitude of movements of the first r 160A relative to movements of the first input device 152A and may require a lower tolerance for unintended motion. Likewise, a relative low scaling factor may allow for a higher tolerance for unintended motion and a corresponding higher first threshold value.

In some embodiments, the first threshold is determined based on a gain setting of the first input device 152A. Similar to the scaling factor, a higher gain factor increases the sensitivity of the first instrument 160A relative to the kinematics of the first input device 152A and may require a lower tolerance for unintended motion. A relative low gain factor may allow for a higher tolerance for unintended motion and a corresponding higher first threshold value.

At 635, the control system 142 performs a mitigation action.

In some embodiments, the mitigation action may include pausing or terminating the first teleoperational control session (e.g., to immediately prevent unintended motion). For example, an operational mode of the computer-assisted system 100 may be changed to alter the behavior of the manipulator assembly 130.

In some embodiments, the mitigating action may include a notification or appropriate indicator (e.g., visual, audible, tactile, etc. style of user interface prompt) directed to the operator. In certain embodiments, a user interface element (e.g., a banner message) regarding the exceeded threshold may be displayed within the field of view displayed to the operator of the computer-assisted system 100. In addition, user interface elements regarding the exceeded threshold may be displayed on one or more other displays (e.g., display unit 154, an external display) of the computer-assisted system 100 (e.g., on a tower display for personnel within an environment of the manipulator assembly 130, on a second input system (e.g., a dual console) communicatively coupled to the manipulator assembly 130, etc.).

In some embodiments, the mitigating action includes an instruction (e.g., conveyed using text and/or images and/or audio) for the operator to exit the first teleoperation control session and reestablish teleoperational control (e.g., in a separate teleoperational control session). For example, establishing a new teleoperational control session may cause the control system 142 to determine an updated transformation between the input device coordinate frame 201 and a workspace coordinate frame based on an updated roll orientation of the imaging device 320.

In some embodiments, the mitigating action includes preventing, by the control system 142, movement of the manipulator assembly 130. For example, movement of one or more instruments 160 may be prevented or at least partially restricted. Furthermore, in some embodiments, the control system 142 may be configured to prevent movement of the first instrument 160A until reestablishing the correct association between the input device 152A and the instrument 160A (e.g., updating the first transformation).

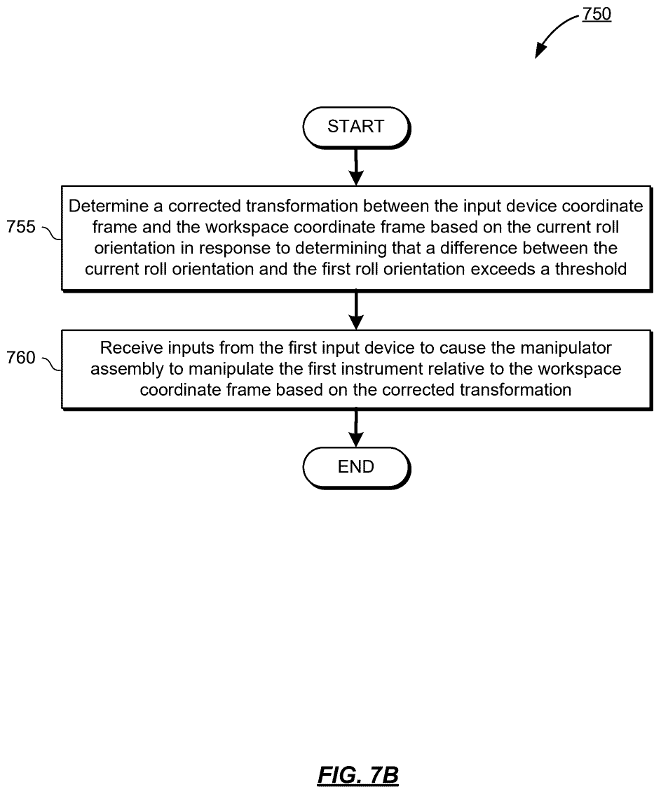

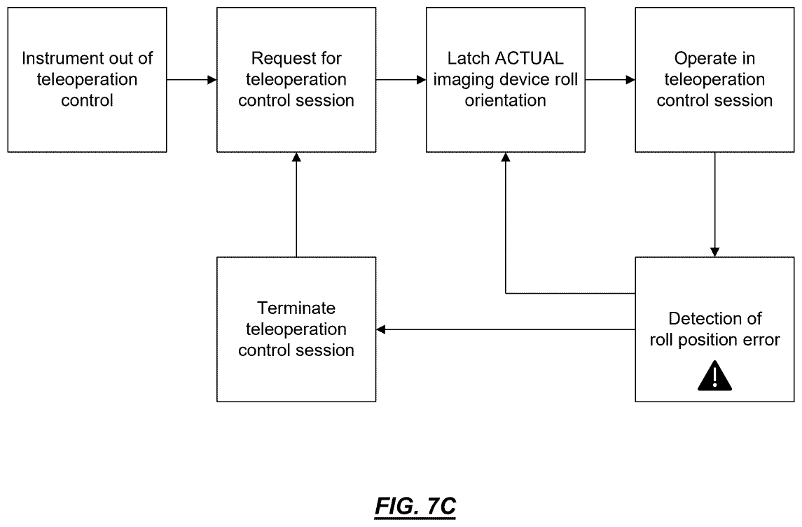

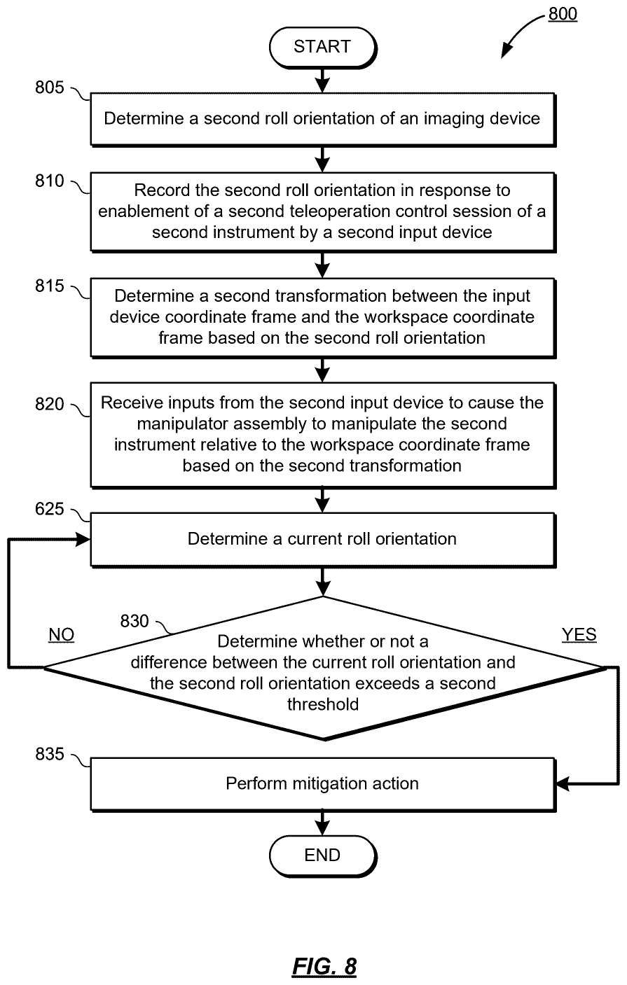

In some embodiments, the control system 142 may take further action to address the difference between the current roll orientation and the first roll orientation, as described in further detail below with respect to FIGS. 7A-7C.

FIGS. 7A-7C show methods according to one or more embodiments.

In FIG. 7A, the control system 142 addresses the difference between the current roll orientation and the first roll orientation by terminating the first teleoperational control session and enabling a separate teleoperational control session with an updated transformation that correctly accounts for the configuration of the imaging device 320 (i.e., an updated roll orientation).

At 705, the control system 142 determines an updated roll orientation of the imaging device. Similar to 605, the updated roll orientation of the imaging device 320 may be determined based on one or more sensors (e.g., as motor encoders, rotary or linear joint encoders) of the manipulator assembly 130 that are associated with actuators and/or joints that support the imaging device 320. The control system 142 may determine the updated roll orientation directly from a signal or information from the one or more sensors or may calculate the updated roll orientation based on configuration information of the manipulator assembly 130.

At 710, the control system 142 records the updated roll orientation of the imaging device in response to enablement of a separate teleoperational control session of the first instrument 160A by the first input device 152A.

In some embodiments, the control system 142 enables the separate teleoperational control session based on a matching grip configuration of the first input device 152A and the first instrument 160A (e.g., the configuration of the first instrument 160A and/or an end effector). The updated roll orientation of the imaging device 320 may be determined during an operation to match the orientation of the first input device 152A in the input device coordinate frame 201 to an orientation of the first instrument 160A in the workspace coordinate frame W.

At 715, the control system 142 determines an updated transformation between the input device coordinate frame 201 and the workspace coordinate frame W based on the updated roll orientation of the imaging device 320. Based on the updated transformation, a movement vector M in the input device coordinate frame 201 corresponds to a movement vector T in the workspace coordinate frame W that is aligned with the updated roll orientation of the imaging device 320.

At 720, the control system 142 receives inputs from the first input device 152A to cause the manipulator assembly 130 to manipulate the first instrument 160A relative to the workspace coordinate frame W based on the updated transformation.

Therefore, in methods 600 and 700, the control system 142 is configured to detect a problem with the first transformation of the first teleoperational control session, terminate the first teleoperational control session before the non-intuitive control scheme can result in an unintended movement, and enable a separate teleoperational control session to recreate an intuitive synchronized control scheme of the manipulator assembly 130 based on an updated transformation.

In FIG. 7B, the control system 142 addresses the difference between the current roll orientation and the first roll orientation by correcting the first transformation without terminating the first teleoperational control session.

At 755, the control system 142 determines a corrected transformation between the

input device coordinate frame 201 and the workspace coordinate frame W based on the current roll orientation in response to determining that the difference between the current roll orientation and the first roll orientation exceeds the first threshold.

At 760, the control system 142 receives inputs from the first input device 152A to cause the manipulator assembly 130 to manipulate the first instrument 160A relative to the workspace coordinate frame W based on the corrected transformation.

Therefore, in methods 600 and 750, the control system 142 is configured to detect a problem with the first transformation of the first teleoperational control session, correct the transformation in situ, and maintain use of the manipulator assembly 130 in an intuitive and synchronized manner, without significant disruption to the operator.

In some embodiments, the control system 142 may switch between method 700 and method 750 based on one or more conditions. For example, the control system 142 may switch to method 750 only under conditions in which there is sufficient computational resources (e.g., processing, memory, bandwidth resources) available to the computer-assisted system 100 to determine the corrected transformation within a threshold time limit. For example, if the amount of time to determine and implement the corrected transformation exceeds the threshold time limit, the control system 142 will terminate the first teleoperational control session to avoid a noticeable loss of control during the first teleoperational control session.

The threshold time limit may be determined based on an input value by the operator or stored information. In some embodiments, the threshold time limit is based on a reaction speed of the operator (e.g., of a specific operator, an average from multiple operators).

FIG. 7C shows the overall work flow of the methods 600, 700, 750, in accordance with embodiments described above.

FIG. 8 shows a method 800 according to one or more embodiments.

In method 800, the control system 142 is configured to simultaneously manage multiple teleoperational control sessions for potential non-intuitive control schemes. In some embodiments, the control system 142 may be configured to independently manage different teleoperational control sessions (e.g., with different thresholds or tolerances).

In some embodiments, the control system 142 is configured to manage a first input device 152A, according to method 600 described above, and to also manage a second input device 152B, according to method 800 as follows, in parallel. The relative timing of methods 600, 800 for the two distinct input devices 152A, B, respectively, may be independent of each other (e.g., handled by independent computing kernels).

At 805, the control system 142 determines a second roll orientation of the imaging device 320. The second roll orientation of the imaging device 320 may be determined separately from the first roll orientation because the input devices 152A, B may be operated independently from each other. For example, the input devices 152A, B may correspond to right and left handles 200 of the user interface system 120 (e.g., a console) of the computer-assisted system 100. Alternatively, the input devices 152A, B may correspond to separate control stations of the computer-assisted system 100 corresponding to independent manipulator arms 150 on separate carts.

At 810, the control system 142 records the second roll orientation of the imaging device 320 in response to enablement of a second teleoperational control session of a second instrument 160B by the second input device 152B. Because the first teleoperational control session and the second teleoperational control session correspond to different input devices 152A, B and different instruments 160A, B, the relative timing for enablement may be different (e.g., the second input device 152B is synchronized some amount of time after the first input device 152A). Accordingly, the second roll orientation and the first roll orientation may be different and therefore may be recorded separately.

In some embodiments, the control system 142 enables the second teleoperational control session based on a matching grip configuration of the second input device 152B and the second instrument 160B (e.g., the configuration of the second instrument 160B and/or an end effector). Because the second roll orientation is an important element of correctly determining and applying a second transformation to the second teleoperational control session, the second roll orientation of the imaging device 320 may be determined and/or recorded upon successfully matching the grip configuration.

At 815, the control system 142 determines a second transformation between the input device coordinate frame 201 and a workspace coordinate frame W based on the second roll orientation of the imaging device 320. The second transformation includes one or more parameters/equations that convert that a movement vector M in the input device coordinate frame 201 to a movement vector T in the workspace coordinate frame W that is aligned with the view coordinate frame 324 of the imaging device 320.

At 820, the control system 142 receives inputs from the second input device 152B to cause the manipulator assembly 130 to manipulate the second instrument 160B relative to the workspace coordinate frame W based on the second transformation.

As discussed above, at 625, the control system 142 determines a current roll orientation of the imaging device 320. In method 800, the current roll orientation may be obtained by multiple processing entities of the control system 142, in parallel (e.g., corresponding to the different teleoperational control sessions).

At 830, the control system 142 determines whether or not a difference between the current roll orientation and the second roll orientation exceeds a second threshold. When the determination is YES (i.e., the difference between the current roll orientation and the second roll orientation exceeds the second threshold), the method continues to 835. When the determination is NO (i.e., the difference between the current roll orientation and the second roll orientation is equal to or less than the second threshold), the method returns to 625 (e.g., immediately or after a delay).

At 830, the control system 142 performs another mitigating action (e.g., a mitigating action as described above, directed to the second input device 152B, the second instrument 160B, and/or the second teleoperational control session).

In some embodiments, the control system 142 terminates the second teleoperational control session in response to a difference between the current roll orientation and the second roll orientation exceeding a second threshold. Because the first teleoperational control session and the second teleoperational control session correspond to different input devices 152A, B and different instruments 160A, B, the relative timing for enablement may be different (e.g., the second input device 152B is synchronized some amount of time after the input device 152A). Accordingly, the second threshold corresponding to the input device 152B and the first threshold corresponding to the first input device 152A may be different. Therefore, the different teleoperational control sessions (e.g., the first teleoperational control session of the first instrument 160A by the first input device 152A and the second teleoperational control session of the second instrument 160B by the second input device 160B) may be independently monitored and terminated based on separate criteria that define the respective thresholds.

Therefore, in method 800, the control system 142 is configured to prevent a non-intuitive control scheme or unintended motion in one or both teleoperational control sessions in an independent manner to minimize impact on the operator and/or an ongoing procedure. While method 800 is directed to managing two teleoperational control sessions, it will be appreciated that the concept of monitoring the roll orientation of the imaging device 320 may be applied to other configurations (e.g., any number of teleoperational control sessions separated into any number of groups with separate thresholds).

Although methods 600, 700, 750, 800 have been described with respect to a limited number of examples and operations, those skilled in the art, having benefit of this disclosure, will appreciate that various other embodiments may be devised without departing from the scope of the present disclosure.

Furthermore, while the various blocks in the flowcharts are presented and described sequentially, one of ordinary skill in the art will appreciate that some or all of the blocks may be executed in different orders, combined, omitted, and some or all of the blocks may be executed in parallel.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed is:

1. A computer-assisted system comprising:
a manipulator assembly configured to support a first instrument and an imaging device;
a first input device configured to be manipulated by an operator relative to an input device coordinate frame; and
a control system communicatively coupled to the first input device and the manipulator assembly, the control system being configured to:
determine a first roll orientation of the imaging device;
record the first roll orientation of the imaging device in response to enablement of a first teleoperational control session of the first instrument by the first input device; and
during the first teleoperational control session:
determine a first transformation between the input device coordinate frame and a workspace coordinate frame based on the first roll orientation of the imaging device;
receive inputs from the first input device to cause the manipulator assembly to manipulate the first instrument relative to the workspace coordinate frame based on the first transformation;
determine a current roll orientation of the imaging device;
determine a difference between the current roll orientation and the first roll orientation;
determine whether or not the difference between the current roll orientation and the first roll orientation exceeds a first threshold; and
in response to the difference between the current roll orientation and the first roll orientation exceeding the first threshold, perform a mitigation action.

2. The computer-assisted system of claim 1,
wherein the mitigation action includes terminating, by the control system, the first teleoperational control session.

3. The computer-assisted system of claim 2,
wherein, after terminating the first teleoperational control session, the control system is configured to:
determine an updated roll orientation of the imaging device;
in response to enablement of a second teleoperational control session of the first instrument by the first input device, record the updated roll orientation of the imaging device.

4. The computer-assisted system of claim 3,
wherein the control system is configured to determine the updated roll orientation of the imaging device during an operation to match an orientation of the first input device in the input device coordinate frame to an orientation of the first instrument in the workspace coordinate frame.

5. The computer-assisted system of claim 3,
wherein the control system is configured to:
determine a updated transformation between the input device coordinate frame and a workspace coordinate frame based on the updated roll orientation of the imaging device;
receive inputs from the first input device to cause the manipulator assembly to manipulate the first instrument relative to the workspace coordinate frame based on the updated transformation.

6. The computer-assisted system of claim 1,
wherein the mitigating action includes:
a visual indicator on a display of the computer-assisted system; or
an instruction for the operator to exit the first teleoperation control session and reestablish teleoperational control.

7. The computer-assisted system of claim 1,
wherein the mitigating action includes preventing, by the control system, movement of the manipulator assembly.

8. The computer-assisted system of claim 1,
wherein the control system is configured to determine the first roll orientation of the imaging device at the time of or during the process of enabling the first teleoperational session.

9. The computer-assisted system of claim 8,
wherein the control system is configured to determine the first roll orientation of the imaging device during an operation to match an orientation of the first input device in the input device coordinate frame to an orientation of the first instrument in the workspace coordinate frame.

10. The computer-assisted system of claim 1, further comprising:
a second input device configured to be manipulated by the operator relative to the input device coordinate frame,
wherein the manipulator assembly is configured to support a second instrument;
wherein the control system is configured to:
determine a second roll orientation of the imaging device;
record the second roll orientation of the imaging device in response to enablement of a second teleoperational control session of the second instrument by the second input device; and
during the second teleoperational control session:
determine a second transformation between the input device coordinate frame and a workspace coordinate frame based on the second roll orientation of the imaging device;
receive inputs from the second input device to cause the manipulator assembly to manipulate the second instrument relative to the workspace coordinate frame based on the second transformation;
determine a difference between the current roll orientation and the second roll orientation;
determine whether or not the difference between the current roll orientation and the second roll orientation exceeds a second threshold; and
in response to the difference between the current roll orientation and the second roll orientation exceeding the second threshold, perform another mitigating action.

11. The computer-assisted system of claim 10,
wherein the control system is configured to independently terminate different teleoperational control sessions for the first instrument and the second instrument.

12. The computer-assisted system of claim 10,
wherein the second threshold is the same as the first threshold.

13. The computer-assisted system of claim 1,
wherein the first threshold is an angular value greater than 10 degrees.

14. The computer-assisted system of claim 1,
wherein the control system is configured to determine the first roll orientation and the current roll orientation based on one or more sensors in a portion of the manipulator assembly that supports the imaging device.

15. The computer-assisted system of claim 14,
wherein the control system is configured to determine the first roll orientation and the current roll orientation by obtaining an encoder value from the one or more sensors.

16. The computer-assisted system of claim 14,
wherein the control system is configured to calculate the first roll orientation and the current roll orientation based on configuration information of the manipulator assembly.

17. The computer-assisted system of claim 14,
wherein the control system is configured to monitor the current roll orientation during the first teleoperational control session.

18. The computer-assisted system of claim 17,
wherein the control system is configured to continuously monitor the current roll orientation based on a processing rate of a processing kernel associated with a portion of the manipulator assembly that supports the imaging device.

19. A method of operating a computer-assisted system including a manipulator assembly configured to support a first instrument and an imaging device, a first input device configured to be manipulated by an operator relative to an input device coordinate frame, and a control system communicatively coupled to the first input device and the manipulator assembly, the method comprising:
determining a first roll orientation of the imaging device;
recording the first roll orientation of the imaging device in response to enablement of a first teleoperational control session of the first instrument by the first input device; and
during the first teleoperational control session:
determining a first transformation between the input device coordinate frame and a workspace coordinate frame based on the first roll orientation of the imaging device;
receiving inputs from the first input device to cause the manipulator assembly to manipulate the first instrument relative to the workspace coordinate frame based on the first transformation;
determining a current roll orientation of the imaging device;
determining a difference between the current roll orientation and the first roll orientation;
determine whether or not the difference between the current roll orientation and the first roll orientation exceeds a first threshold; and
in response to the difference between the current roll orientation and the first roll orientation exceeding the first threshold, performing a mitigating action.

20. A non-transitory machine-readable medium comprising a plurality of machine-readable instructions executed by one or more processors associated with a computer-assisted system including a manipulator assembly configured to support a first instrument and an imaging device, a first input device configured to be manipulated by an operator relative to an input device coordinate frame, and a control system communicatively coupled to the first input device and the manipulator assembly, the plurality of machine-readable instructions causing the one or more processors to:
determine a first roll orientation of the imaging device;
record the first roll orientation of the imaging device in response to enablement of a first teleoperational control session of the first instrument by the first input device; and
during the first teleoperational control session:
determine a first transformation between the input device coordinate frame and a workspace coordinate frame based on the first roll orientation of the imaging device;
receive inputs from the first input device to cause the manipulator assembly to manipulate the first instrument relative to the workspace coordinate frame based on the first transformation;
determine a current roll orientation of the imaging device;
determine a difference between the current roll orientation and the first roll orientation;
determine whether or not the difference between the current roll orientation and the first roll orientation exceeds a first threshold; and
in response to the difference between the current roll orientation and the first roll orientation exceeding the first threshold, perform a mitigating action.
